(19) United States
(12) Patent Application Publication
(10) Pub. No.: US 2011/0086012 A1
(43) Pub. Date: Apr. 14, 2011

(54) Rejuvenation Method

(76) Inventor: Yu-Han Wang, Fanlu Township (TW)

(21) Appl. No.: 12/577,973

(22) Filed: Oct. 13, 2009

(51) Int. Cl. A61K 35/14; A61K 35/12

(52) U.S. Cl. 424/93.73; 424/93.7

(57) ABSTRACT

A rejuvenation method includes a) removing blood or tissue from a human body or animal body, b) nurturing and purifying the blood or tissue, and c) implanting the blood or tissue that has been nurtured and purified into a user to increase the amount of stem cells in the user and to recover degraded organs in the user. Thus, the rejuvenation method is used to increase the number of the stem cells in the user successively so that the degraded organs in the user are repaired and recovered completely and can be returned to the optimum state so as to achieve the rejuvenation purpose. In addition, the blood or tissue is removed and taken from the user's body without causing a rejection.

### BACKGROUND OF THE INVENTION

1. Field of the Invention

The present invention relates to a method and more particularly, to a rejuvenation method which removes blood or tissue from a human body or animal body, then nurtures and purifies the blood or tissue, and then implants the blood or tissue that has been nurtures and purified into a user to increase the amount of stem cells in the user and to recover degraded organs in the user.

2. Description of the Related Technologies

When the age of the human being is increased, the organs of the human being are easily degraded or sick. However, there is no effective method to prevent the organ degradation of the human being and to recover the organs of the human being to the optimum state.

### BRIEF SUMMARY OF THE INVENTION

In accordance with the present invention, it provides a rejuvenation method, comprising a) removing blood or tissue from a human body or animal body, b) nurturing and purifying the blood or tissue, and c) implanting the blood or tissue that has been cultivated and purified into a user to increase the amount of stem cells in the user and to recover degraded organs in the user.

The primary objective of the present invention is to provide a rejuvenation method that is used to increase the number of the stem cells in the user successively so that the degraded organs in the user are repaired and recovered completely and can be returned to the optimum state so as to achieve the rejuvenation purpose.

Another objective of the present invention is to provide a rejuvenation method, wherein the blood or tissue is removed and taken from the user's body.

### BRIEF DESCRIPTION OF THE SEVERAL VIEWS OF THE DRAWING(S)

FIG. 1 is a flow chart of a rejuvenation method in accordance with the preferred embodiment of the present invention.

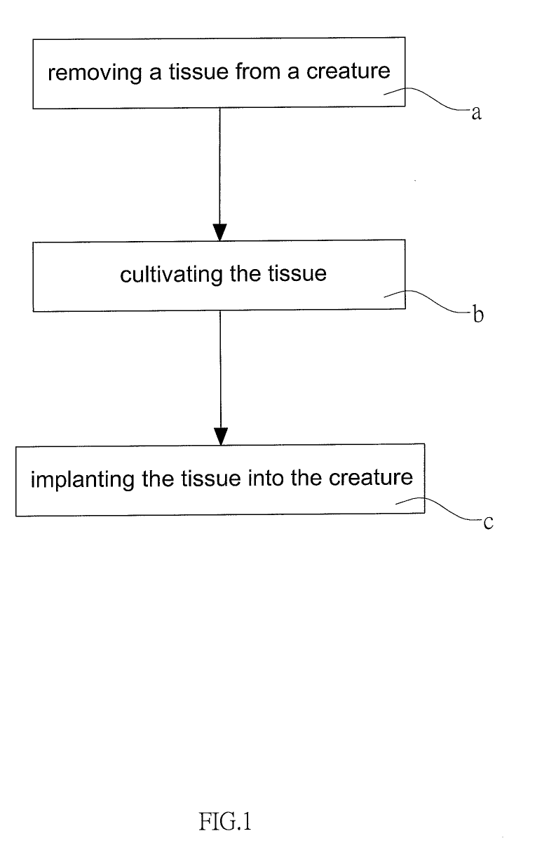

### DETAILED DESCRIPTION OF THE INVENTION

A rejuvenation method in accordance with the preferred embodiment of the present invention comprises a) removing blood or tissue from a human body or animal body, b) nurturing and purifying the blood or tissue, and c) implanting the blood or tissue that has been nurtured and purified into a user to increase the amount of stem cells in the user and to recover degraded organs in the user.

In such a manner, the rejuvenation method is recycled successively to increase the number of the stem cells in the user successively so that the degraded organs in the user are recovered completely and are returned to the optimum state so as to achieve the rejuvenation purpose.

Accordingly, the rejuvenation method is used to increase the number of the stem cells in the user successively so that the degraded organs in the user are repaired and recovered completely and can be returned to the optimum state so as to achieve the rejuvenation purpose. In addition, the blood or tissue is removed and taken from the user's body.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A rejuvenation method, comprising:
a) Removing blood or tissue from a human body or animal body;
b) Nurturing and purifying the blood or tissue;
c) Implanting the blood or tissue that has been nurtured and purified into a user to increase the amount of stem cells in the user and to recover degraded organs in the user.
